(19) United States
(12) Patent Application Publication
(10) Pub. No.: US 2008/0161564 A1
(43) Pub. Date: Jul. 3, 2008

(54) Aromatic sulfonated ketals

(76) Inventors: Mark A. Schwindt, Boulder, CO (US); Robert J. Topping, Longmont, CO (US); Charles E. Tucker, Superior, CO (US)

Correspondence Address: ROCHE PALO ALTO LLC;Patent Law Department, M/S A2-250, 3431 Hillview Avenue, Palo Alto, CA, 94304, US

(21) Appl. No.: 12/004,204

(22) Filed: Dec. 20, 2007

Related U.S. Application Data
(60) Provisional application No. 60/877,788, filed on Dec. 29, 2006.

(51) Int. Cl. C07D 241/10

(52) U.S. Cl. 544/336

(57) ABSTRACT

The present invention advantageously provides ketal functional compounds that can be strong electrophiles under conditions compatible with ketal groups, are stable, crystalline solids at room temperature, and are much safer to handle than ketal iodides. The present invention accomplishes by incorporating aromatic sulfonyl moieties into ketal functional materials. The compounds are useful starting materials or intermediates in the synthesis of more complex organic molecules.

### PRIORITY CLAIM

The present non-provisional patent Application claims benefit from U.S. Provisional Patent Application having Ser. No. 60/877,788, filed on Dec. 29, 2006, by Topping et al., and titled AROMATIC SULFONATED KETALS, wherein the entirety of said provisional patent application is incorporated herein by reference.

### FIELD OF THE INVENTION

The present invention relates to aromatic sulfonated ketals, methods of making these materials, and methods of using these materials in the synthesis of more complex molecules such as pharcologically active molecules. The ketals include aromatic sulfonyl moieties so that representative embodiments are stable, crystalline solids at room temperature.

### BACKGROUND OF THE INVENTION

Glucokinase (GK) is one of four hexokinases that are found in mammals [Colowick, S. P., in The Enzymes, Vol. 9 (P. Boyer, ed.) Academic Press, New York, N.Y., pages 1-48, 1973]. The hexokinases catalyze the first step in the metabolism of glucose, i.e., the conversion of glucose to glucose-6-phosphate. Glucokinase has a limited cellular distribution, being found principally in pancreatic β-cells and liver parenchymal cells. In addition, GK is a rate-controlling enzyme for glucose metabolism in these two cell types that are known to play critical roles in whole-body glucose homeostasis [Chipkin, S. R., Kelly, K. L., and Ruderman, N. B. in Joslin's Diabetes (C. R. Khan and G. C. Wier, eds.), Lea and Febiger, Philadelphia, Pa., pages 97-115, 1994]. The concentration of glucose at which GK demonstrates half-maximal activity is approximately 8 mM. The other three hexokinases are saturated with glucose at much lower concentrations (<1 mM).

Therefore, the flux of glucose through the GK pathway rises as the concentration of glucose in the blood increases from fasting (5 mM) to postprandial (≈10-15 mM) levels following a carbohydrate-containing meal [Printz, R. G., Magnuson, M. A., and Granner, D. K. in Ann. Rev. Nutrition Vol. 13 (R. E. Olson, D. M. Bier, and D. B. McCormick, eds.), Annual Review, Inc., Palo Alto, Calif., pages 463-496, 1993]. These findings contributed over a decade ago to the hypothesis that GK functions as a glucose sensor in β-cells and hepatocytes (Meglasson, M. D. and Matschinsky, F. M. Amer. J. Physiol. 246, E1-E13, 1984).

In recent years, studies in transgenic animals have confirmed that GK does indeed play a critical role in whole-body glucose homeostasis. Animals that do not express GK die within days of birth with severe diabetes while animals overexpressing GK have improved glucose tolerance (Grupe, A., Hultgren, B., Ryan, A. et al., Cell 83, 69-78, 1995; Ferrie, T., Riu, E., Bosch, F. et al., FASEB J., 10, 1213-1218, 1996). An increase in glucose exposure is coupled through GK in β-cells to increased insulin secretion and in hepatocytes to increased glycogen deposition and perhaps decreased glucose production.

The finding that type II maturity-onset diabetes of the young (MODY-2) is caused by loss of function mutations in the GK gene suggests that GK also functions as a glucose sensor in humans (Liang, Y., Kesavan, P., Wang, L. et al., Biochem. J. 309, 167-173, 1995). Additional evidence supporting an important role for GK in the regulation of glucose metabolism in humans was provided by the identification of patients that express a mutant form of GK with increased enzymatic activity. These patients exhibit a fasting hypoglycemia associated with an inappropriately elevated level of plasma insulin (Glaser, B., Kesavan, P., Heyman, M. et al., New England J. Med. 338, 226-230, 1998). While mutations of the GK gene are not found in the majority of patients with type II diabetes, compounds that activate GK, and thereby increase the sensitivity of the GK sensor system, would still be useful in the treatment of the hyperglycemia characteristic of all type II diabetes. Glucokinase activators would increase the flux of glucose metabolism in β-cells and hepatocytes, which would be coupled to increased insulin secretion. Such agents would be useful for treating type II diabetes.

The following glucokinase activator (referred to herein as the compound of Formula I)

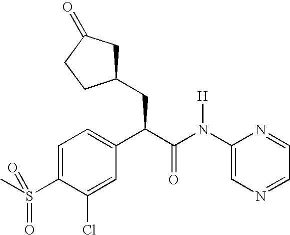

and its isopropanol (IPA) solvate of the formula:

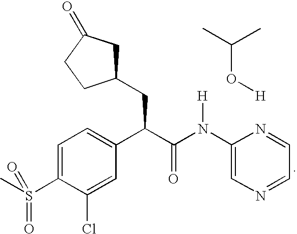

are under evaluation as a potentially new therapy for the treatment of Type 2 diabetes. The compound of Formula I has also been described in PCT Patent Publication No. WO 03/095438 as well as in the co-pending U.S. application Ser. No. 11/583,971, corresponding to U.S. Publication No. 2007/0129554, titled ALPHA FUNCTIONALIZATION OF CYCLIC, KETALIZED KETONES AND PRODUCTS THEREFROM, bearing Attorney Docket No. RCC0021/US, and filed Oct. 19, 2006, in the names of Harrington et al (hereinafter Application A); U.S. Provisional Application No. 60/791,256 titled PROCESS FOR THE PREPARATION OF A GLUCOKINASE ACTIVATOR, bearing Attorney Docket No. 23026, and filed Apr. 12, 2006 in the names of Andrzej Robert Daniewski et al., (hereinafter Application B); and U.S. Provisional Patent Application No. 60/877,877, titled EPIMERIZATION METHODOLOGIES FOR RECOVERING STEREOISOMERS IN HIGH YIELD AND PURITY, bearing Attorney Docket No. RCC0030/P1, and filed Dec. 29, 2006 in the name of inventor Robert J. Topping (hereinafter Application C). All of these patent documents are incorporated herein by reference in their respective entireties for all purposes.

Application B schematically shows and describes a multi-step reaction scheme in which the compound of Formula I and its IPA (isopropyl alcohol) is manufactured from a ketal acid starting material in nine main reaction steps. Step five of this synthesis involves using a ketal iodide to alkylate an aromatic ester to form a mixture of epimers. Because neither the ketal acid or the ketal alcohol are conveniently converted directly to the iodide, the conversion to the iodide occurs through the intermediate mesylate. It is plausible that Application B could have developed conditions to make the mesylate work for this alkylation as well. However, alkylation conditions that work for the iodide are not appropriate for use of a mesylate, nor for a tosylate which is an aspect of the present invention described below.

However, there are drawbacks to the synthesis scheme shown in Application B. First, both the ketal mesylate and the ketal iodide are oils. Being oils, both compounds are hard to isolate and purify, and the reaction scheme is relatively difficult to scale up for large scale production. The iodide also suffers from a short shelf life. This instability as well as toxicity concerns associated with the iodide require careful handling and attention to safety protocols.

Accordingly, it would be very desirably to uncover ketal intermediates that are strong electrophiles under conditions compatible with ketal groups; are solids at room temperature for easier handling, purification, and isolation; and are stable and less toxic than ketal iodides to easy handling and safety concerns.

### SUMMARY OF THE INVENTION

The present invention advantageously provides ketal functionalized compounds that can be sufficiently strong electrophiles under conditions compatible with ketal groups; are stable, crystalline solids at room temperature; and are much safer to handle than ketal iodides. The present invention accomplishes this by incorporating aromatic sulfonyl moieties into ketal functional materials. The compounds are useful starting materials or intermediates in the synthesis of more complex organic molecules.

According to one representative use, aromatic sulfonated ketals of the present invention can be used in the synthesis of the compound of Formula I. For example, a ketal acid is readily converted to a ketal alcohol. The ketal alcohol, in turn, is readily converted to a ketal including an aromatic sulfonate moiety. This can then be directly used in alkylation without having to proceed via a mesylate or an iodide. Yield and purity of the compound of Formula I are enhanced.

In one aspect, the present invention relates to an aromatic sulfonated ketal. The ketal includes:
a sulfonate moiety of the formula —O—S(O)(O)—, wherein there is a double bond between each oxygen in parentheses and the sulfur atom;
a ketal moiety linked to the oxygen atom with an available valent site of the sulfonate moiety by a first linkage; and
an aromatic moiety coupled to the S of the sulfonate moiety by a second linkage.

In another aspect, the present invention relates to a method of making an aromatic sulfonated ketal. A ketal alcohol (or amine) is provided. The alcohol (or amine) is reacted with a co-reactant comprising a source of an aromatic sulfonyl moiety under conditions effective to convert the alcohol moiety of the ketal to an aromatic sulfonated moiety.

In another aspect, the present invention relates to a method of using an aromatic ketal sulfonate. The aromatic sulfonated ketal is reacted with an aromatic ester in the presence of a base, wherein the aromatic ester comprises:
a carbon atom that is in an alpha position relative to a —C(O)— moiety;
an aromatic moiety is linked to the alpha carbon atom; and
wherein at least one remaining substituent of the alpha carbon atom is H.

### BRIEF DESCRIPTION OF THE DRAWINGS

The above mentioned and other advantages of the present invention, and the manner of attaining them, will become more apparent and the invention itself will be better understood by reference to the following description of the embodiments of the invention taken in conjunction with the accompanying drawings, wherein:

FIG. 1 shows an illustrative reaction synthesis for preparing an aromatic ketal sulfonate from ketal acid, salt, or ester starting materials.

FIG. 2 shows a more preferred reaction synthesis for preparing an aromatic ketal sulfonate from ketal acid, salt, or ester starting materials.

FIG. 3a shows one embodiment of a chiral (S), nitrogen containing cation useful in the practice of the present invention.

FIG. 3b shows one embodiment of a chiral (R), nitrogen containing cation useful in the practice of the present invention.

FIG. 4 shows an illustrative reaction in which aromatic ketal sulfonates of the present invention are used in an alkylation reaction.

FIG. 5 shows an illustrative synthesis scheme for making the compound of Formula I and its IPA solvate in which principles of the present invention are used.

### DETAILED DESCRIPTION

The embodiments of the present invention described below are not intended to be exhaustive or to limit the invention to the precise forms disclosed in the following detailed description. Rather the embodiments are chosen and described so that others skilled in the art may appreciate and understand the principles and practices of the present invention.

In one aspect, the present invention relates to aromatic sulfonated ketals, methods of making these compounds, and uses of these compounds. In the practice of the present invention, aromatic sulfonated ketals are compounds comprising at least one sulfonate moiety, a ketal moiety linked to the sulfonate moiety via a first linkage that includes a suitable linking group, and an aromatic moiety linked to the sulfonate moiety by a second linkage that involves a single bond or a suitable linking group.

A sulfonyl moiety refers to a divalent moiety of the formula —S(O)(O)—, wherein there is a double bond between each oxygen and the sulfur atom. Under some conventional understandings, the term sulfonyl indicates that the moiety is obtained from a sulfonic acid moiety. However, the term sulfonyl as used in the present invention applies to any —S(O)(O)— moiety regardless of the method used to form the moiety. A sulfonate moiety refers to a divalent moiety of the formula —O—S(O)(O)—. Each oxygen in parenthesis is coupled to the S by a double bond. The third oxygen is coupled to the S by a single bond and has a remaining valent site to bond with another moiety. Also, the S has a remaining valent site, too.

A ketal moiety is a functional group that includes a carbon atom bonded to both —OZ¹ and —OZ² groups, wherein each of Z¹ and Z² independently may be a wide variety of monovalent moieties or co-members of a ring structure. In representative embodiments, Z¹ and Z² alone or as co-members of a ring structure are linear, branched, or cyclic alkyl(ene); preferably alkyl(ene) of 1 to 15, preferably 2 to 5 carbon atoms. The divalent, branched alkylene backbone associated with neopentyl glycol is a preferred structure when Z¹ and Z² are co-members of a ring structure.

A ketal is structurally equivalent to an acetal, and sometimes the terms are used interchangeably. In some uses, a difference between an acetal and a ketal derives from the reaction that created the group. Acetals traditionally derive from the reaction of an aldehyde and excess alcohol, whereas ketals traditionally derive from the reaction of a ketone with excess alcohol. For purposes of the present invention, though, the term ketal refers to a molecule having the resultant ketal/acetal structure regardless of the reaction used to form the group.

The linking group that couples the ketal to the sulfonate moiety generally is trivalent. One valent site is needed to couple to the S of the sulfonyl moiety, while two valent sites are used to couple to the carbon atom of the ketal that is bonded to the OZ¹ and OZ² groups. The linking group may be saturated or unsaturated, chiral or achiral. The linking group desirably may be aliphatic, aromatic, or a combination of aliphatic and aromatic. Often, the C atom of the ketal and at least a portion of the linking group form a cyclic structure. This cyclic moiety may then be coupled to the S of the sulfonyl via a single bond or a suitable divalent linking group. Often, the divalent linking group includes at least a hetero atom adjacent to the S of the sulfonyl moiety. Examples of hetero atoms include S, O, N, P, Si, combinations of these and the like. Desirably, the hetero atom adjacent the S of the sulfonyl is oxygen. Such structures are readily formed from alcohol precursors, as will be described in more detail below.

The aromatic moiety may be any substituted or non-substituted moiety that includes at least one aromatic ring structure and may be chiral or achiral. Optionally, the aromatic moiety may include aliphatic portions. The aromatic ring structure may be fused or non-fused with respect to other aromatic or aliphatic ring structures (e.g., as when two substituents of any such aromatic ring are co-members of a ring structure). The aromatic moiety optionally may incorporate one or more hetero atoms such as O, P, S, Si, N and/or the like as constituents and/or substituents of aromatic or aliphatic moieties incorporated into the aromatic moiety.

The linking group that links the aromatic moiety to the S of the sulfonate moiety desirably may be a single bond or any saturated or unsaturated divalent moiety. The linking group may be substituted or unsubstituted, saturated or unsaturated, chiral or achiral. The linking group can be linear, cyclic or branched. The linking group optionally may incorporate one or more hetero atoms such as O, P, S, N, Si, and/or the like. Preferably, the linking group is a single bond or is a linear, branched or cyclic alkylene radical containing from 1 to 15 carbon atoms, preferably 1 to 5, more preferably 1 to 2 carbon atoms. Most preferably, the linking group is a single bond or an alkylene group of 1 to 6 carbon atoms such as —CH₂—.

According to one methodology, an aromatic ketal sulfonate of the present invention is derived from a ketal alcohol precursor and a co-reactant that serves as a source of an aromatic sulfonyl moiety. When reacted together, the sulfonyl moiety is converted to a sulfonate moiety. The ketal alcohol itself is conveniently derived from a ketal acid or a salt or ester of a ketal acid. An illustrative synthesis begins by providing a ketal acid, salt, or ester. If the ketal material is first provided in the form of a salt or acid, it is first desirable to convert the salt or ester to an acid form. Direct reduction of the salt could tend to generate undesirable by-products such as the corresponding free amine. Converting the salt or ester to an acid would contaminate the resultant ketal-alcohol during workup, and the amine could be very difficult to separate at that point. This may be accomplished using any conventional technique.

For instance, when the ketal is supplied in a salt form, an acid may be used for salt cleavage. One way to accomplish this is to disperse the salt in a suitable organic solvent that is immiscible with water. It is convenient if the solvent is the same as the organic solvent to be used for reduction. Toluene is one illustrative solvent that may be used for both the salt cleavage and the reduction. It is also convenient to use aqueous acid. The aqueous acid may be added to the salt containing mixture or the ketal can be slowly added to the aqueous acid. In any case, mixing occurs with agitation. The resultant two-phase mixture is allowed to settle. The ketal acid product will tend to be more soluble in the organic phase, while salt by-products will tend to be in the aqueous phase. The two phases are easily separated to recover the ketal acid in the organic phase. Optionally, the organic phase can be washed one or more additional times with water and/or the aqueous phase can be washed one or more additional times with organic solvent, to further enhance the purity and yield of the ketal acid. At the end of any such washes, the organic phases containing the ketal acid can be combined, optionally concentrated, optionally isolated, and then taken forward to carry out the reduction reaction.

The acid used for salt or ester cleavage to yield the ketal acid should be of moderate strength. If the acid is too strong, the acid could degrade the ketal moiety. Examples of suitable acids of moderate strength that are reasonably compatible with the ketal group include organic acids such as citric acid, acetic acid, succinic acid, tartaric acid, malonic acid, malic acid, and combinations of these, and the like. Desirably, only enough acid is added to ensure that cleavage of as much of the salt or ester is achieved as is practical, inasmuch as too much excess acid risks degradation of the ketal group even when using an acid of moderate strength.

In the practice of the present invention, the reduction of the ketal functional carboxylic acid (or salt or ester thereof) occurs in a reaction medium comprising a reducing agent that is a hydride comprising at least one alkoxy moiety and at least one additional constituent. The reducing agent and the ketal alcohol may be combined all at once or more desirably gradually as the reduction reaction progresses. Preferably, the ketal-acid is added to the excess reducing agent.

In representative embodiments, each alkoxy moiety of the reducing agent generally independently has the formula —OR³—, wherein R³ is a divalent aliphatic and/or aromatic hydrocarbyl. Desirably, each R³ is a linear, branched or cyclic alkylene moiety containing 1 to 10 carbon atoms, often 1 to 6 carbon atoms.

The at least one other constituent included in the reducing agent comprises one or more atoms such as B, Li, Na, K, Mg, Ca, Al, selenium, bismuth, antimony, tellurium, silicon, lead, germanium, arsenic, nitrogen, tin, polonium, combinations of these, and the like. These atoms may be present in any suitable form, including as a constituent of an oxygen-containing species.

A particularly preferred kind of reducing agent is a hydride that comprises one or more alkoxy moieties and aluminum in a suitable form such as an aluminate. An example of one such reducing agent is sodium dihydro-bis-(2-methoxyethoxy)aluminate (also referred to as SDMA). SDMA is commercially available under the trade designation VITRIDE in solutions comprising about 69 weight percent of SDMA in toluene from Zeeland Chemicals). The VITRIDE reducing agent has a reductive strength that is somewhat in-between NaBH₄ and LiAlH₄. The VITRIDE material is a readily transferable liquid that is compatible with ketals, and is compatible with common, inexpensive, aprotic solvents such as toluene and the like.

The amount of reducing agent included in the reaction mixture may vary over a wide range. Generally, at least a modest excess of the reducing agent is included to help ensure that as much acid (or salt or ester thereof) is reduced as is practical to maximize yield. Using too much is wasteful of reagent and can make it more difficult to isolate the resultant product from the left over reducing agent. For instance, in the case of the VITRIDE reducing agent, each VITRIDE molecule has two available hydrides on a theoretical basis. Three hydrides are needed to reduce an acid to an alcohol. One hydride deprotonates the acid. A second hydride reduces the carboxylate to an aldehyde. A third hydride reduces the aldehyde to form the alcohol. Therefore, the molar ratio of the VITRIDE reducing agent to the acid is desirably at least 1.5:1 so that there are 3 equivalents of hydride for each equivalent of acid. Using such amounts achieves 95% yield of the ketal alcohol in representative embodiments. Using lesser amounts of the VITRIDE, e.g., 1.2 moles per mole of acid, achieves only 90% yields in other representative embodiments.

Using an excess of the reducing agent will tend to help achieve higher conversion of acid to alcohol. However, using too much excess reducing agent is not desirable, inasmuch as using more would require more quenching reagent. Also, using more would tend to produce more by-products to be removed. Hence, using too much reducing agent would make scale up less cost-efficient. Accordingly, in the case of VITRIDE, any excess of the VITRIDE should be slight, e.g., 1.55 moles of VITRIDE per mole of the acid. In one representative mode of practice according to a larger scale reaction, using 487.8 kg of a 70% solution of SDMA in toluene per 752.4 mol of a ketal acid was found to be suitable.

Because both the desired ketal acid starting material and the reducing agent are both soluble in a wide range of organic, aprotic, nonpolar solvents, the reducing conversion may occur in a wide range of solvents or solvent mixtures. Preferred solvents are aliphatic and/or aromatic hydrocarbon solvents inasmuch as such solvents are widely available and inexpensive. Toluene is a preferred organic solvent as it is widely available and cost effective. Toluene also facilitates aqueous workup after the conversion via conventional extraction techniques to separate the reduction by products from the desired alcohol product. The by-products tend to be more soluble in an aqueous phase, while the ketal alcohol product tends to be more soluble in the organic phase. THF could also be a good solvent, but workup will tend to be harder due to water miscibility.

The amount of solvent used to carry out the conversion of ketal acid to ketal alcohol can vary over a wide range. If there is too little solvent, though, then the intermediate ketal-acid can precipitate prior to the reduction, resulting in processing issues with the reduction. On the other hand, if there is too much solvent, then the reaction may take longer, the cost increases due to higher solvent usage and less vessel utilization. Balancing such concerns, the reaction medium to carry out the reduction generally may include from about 200 to about 1000 liters, more desirably 300 to 700 liters, of solvent per 50 to 500 kilograms of acid (or salt or ester).

The reduction reaction may be carried out at a wide range of temperatures over a wide range of time periods. For instance, the reaction may occur at any temperature ranging from just above 0° C. to 60° C. The reduction of carboxylic acids may be too sluggish (slow) below 0° C. to be practical, and degradation may tend to occur above 60° C. . More desirably, the reaction mixture is maintained slightly chilled or near room temperature such as at a temperature in the range from about 5° C. to about 30° C., more commonly about 20° C. to about 30° C. Conducting the reduction at such moderately higher temperatures is preferred to enhance yield of the ketal alcohol without undue risk of degradation of the reactants or products. The reaction desirably may occur for a time period in the range of from about a few minutes to several hours, desirably about 5 minutes to 8 hours.

After the reduction reaction is complete, it is desirable to quench the reduction reaction. According to one illustrative technique, this is accomplished by adding a base to the reaction mixture or vice versa. It is convenient to use aqueous base such as aqueous NaOH or the like. The ketal is best added slowly added to the aqueous base because adding NaOH (aq) to the reaction could be more dangerous due to less control over hydrogen evolution. Additionally, adding aqueous NaOH to the ketal alcohol mixture tends to result in salt precipitation due to lack of water during the early phases of the quench. In any case, mixing occurs with agitation. The resultant two-phase mixture is allowed to settle. The ketal alcohol product will tend to be more soluble in the organic phase, while salt by-products will tend to be more soluble in the aqueous phase. The two phases are easily separated to recover the ketal alcohol in the organic phase. Optionally, the organic phase can be washed one or more additional times with water and/or the aqueous phase can be washed one or more additional times with organic solvent, to further enhance the purity and yield of the ketal alcohol. At the end of any such washes, the resultant organic phases can be combined, optionally concentrated, optionally isolated, and/or taken forward to carry further desired processing.

The conversion of ketal acid to ketal alcohol tends to yield by-products that can influence the effectiveness of extraction yields when aqueous work up is used to recover the ketal alcohol product in an organic phase, e.g., a toluene phase. For example, in the case of VITRIDE, quenching the reduction reaction tends to liberate 2-methoxyethanol. The presence of this alcohol unfortunately enhances the water solubility of the ketal alcohol product to some degree. Although a major portion of the ketal alcohol product will be present in the organic phase upon aqueous workup, significant portions of the ketal alcohol nonetheless will be solubilized in the aqueous washes used to remove salt by-products.

In short, in order to upgrade the yield of the ketal alcohol, it is desirable to minimize the loss of ketal alcohol into the aqueous washes during aqueous workup. To accomplish this, it is desirable to extract the aqueous phase(s) due to the slight water solubility of the ketal alcohol that is induced by the presence of reduction reaction by-products such as 2-methoxy ethanol. Advantageously, therefore, the present invention uses one or more back extractions of the aqueous phase(s) to recover ketal alcohol from the aqueous phases that otherwise would be lost. Accordingly, after subjecting the alcohol product mixture to a first extraction among an organic phase and an aqueous phase, the aqueous phase can be subjected to one or more organic washes in order to recover additional ketal alcohol from the aqueous phase(s). The upgrade in yield is significant. In representative embodiments, yields of 95% are achieved when using such back extraction methods. In contrast, yields of only 85% are achieved without the back extractions.

Optionally, any of the organic layers obtained from the primary or back extractions can also be washed with water to further upgrade yields and/or purity, although such washes could cause some amounts of ketal alcohol to be lost to the aqueous layers unless such additional aqueous layers are also back extracted with an organic wash such as toluene.

In a second step, the ketal alcohol is reacted with a co-reactant that serves as a source of an aromatic sulfonyl-containing moiety. The sulfonyl moiety is converted to a sulfonate moiety upon reaction with the ketal alcohol. According to one suitable approach for accomplishing this, a protective blanket of nitrogen or the like is maintained over the reaction mixture throughout the following procedures. Initially, a base is dissolved in solvent to provide a basic reaction reagent. One example of a suitable base is 1,4 diazabicyclo[2,2,2]octane (also known as DABCO or TEDA). Aromatic solvents such as toluene or the like are preferred. Other examples of suitable solvents include ethyl acetate, isopropyl acetate, combinations of these, and the like. It is desirable that the base be present in a moderate molar excess relative to the ketal alcohol. If too little base is used, e.g., there is a stoichiometric excess of the ketal alcohol, there could be reactivity and/or impurity issues. Accordingly, a suitable reagent may be prepared by dissolving enough base in the solvent so that the molar ratio of the base to the ketal alcohol to be processed is greater than 1, preferably 1.05 to 4, more preferably about 1.05 to about 1.5. A suitable concentration of base in the solvent may be in the range of from about 100 to about 1000, preferably about 200 to about 500 moles of base per 100 to about 1000 liters of solvent.

Next, a solution containing pre-dissolved ketal alcohol is added to the basic reaction medium. Desirably, this solution includes enough solvent to help ensure that the ketal alcohol is fully dissolved. Greater amounts of solvent may be used, but this adds little benefit and wastes reagent. The resultant mixture is stirred and cooled such as to a temperature from about 0° C. to about 20° C., more typically 0° C. to about 12° C. The solvent is conveniently the same as was used to dissolve the base, e.g., toluene.

A co-reactant that is a source of the aromatic sulfonyl moiety is then added to the cooled reaction medium. In order to help ensure full conversion of the ketal alcohol to an aromatic sulfonated ketal, it is desirable to use a moderate stoichiometric excess of the co-reactant relative to the ketal alcohol to be converted. Generally, using an excess such that the molar ratio of the co-reactant to the ketal alcohol is greater than 1, preferably 1.05 to about 3, more preferably 1.05 to about 1.5 would be suitable. The co-reactant may be pre-dissolved in a solvent. Conveniently, this solvent is the same, e.g., toluene, as is already present in the reaction mixture.

Desirably, the co-reactant solution is added slowly over a period of time with mixing while the mixture is maintained at a cool temperature, e.g., from about 1° C. to about 15° C., preferably about 4° C. to about 11° C. By way of example, the co-reactant solution may be slowly added over a period ranging from about ten seconds to about 8 hours, preferably about 60 seconds to about 4 hours, more preferably about 30 minutes to 1.5 hours. After adding the co-reactant, the mixture may be stirred at the cool temperature, e.g., about 5° C., for a period of time ranging from about 60 seconds to about 6 hours, preferably about 1 to 3 hours. Higher temperatures could be used unless the risk of undue degradation of reactants and/or products were to become an issue. In any event, using cooler temperatures is preferred because the reaction is still fast at the cooler temperatures and the risk of degradation due to thermal effects is minimized.

After this, a reagent such as a dilute sodium bicarbonate solution, e.g., about 8.5% by weight sodium bicarbonate in water, is added to the reaction mixture. The sodium bicarbonate helps to ensure that no acid is present. It also helps to hydrolyze any unreacted aromatic sulfonyl containing material, e.g., tosyl chloride in representative embodiments, to a salt (e.g., sodium tosylate) so that the unreacted tosyl chloride is not present during isolation of the desired product. The bicarbonate may be added all at once or slowly over a brief period of time such as from about 5 seconds to about one hour, preferably from about one minute to about 15 minutes. After adding the bicarbonate, the mixture may be stirred for an additional period of time at a suitable temperature. This additional period of time may occur over a period ranging from about 3 minutes to about 8 hours, preferably about 20 minutes to about 4 hours at a temperature in the range of from about 0° C. to about 30° C., preferably about 20° C. to about 25° C. Ambient temperature would also be suitable and would avoid the expense of heating or chilling the mixture during this additional period.

The mixture then is allowed to settle. The mixture will separate into an organic phase containing the desired aromatic sulfonated ketal product dissolved in an organic solvent such as toluene and an aqueous phase containing by-products such as salts (DABCO hydrochloride, sodium tosylate, excess DABCO). Optionally, the organic layer can be washed one or more additional times with water to further upgrade the yield and purity of the desired product in the organic phase. Unlike the situation with the reduction of ketal acid to form the ketal alcohol, back-extractions of the water washes are not as necessary in the present context since the resultant aromatic sulfonated ketal tends to have very low water solubility in water, unlike the ketal alcohol.

The resultant aromatic sulfonated ketal product may be subjected to conventional work up and isolation procedures. One illustrative work up and isolation procedure involves recovering the product through one or more crystallizations in an organic, aprotic solvent such as heptane in which the ketal product is insoluble. A protective blanket of nitrogen or the like is still maintained over the reagents containing the product. Initially, the organic phase containing the ketal product is separated from any aqueous phases and then concentrated. Then, the crystallization solvent, such as heptane, is added to the mixture containing the ketal product. The crystallization solvent may be added slowly with mixing over a period of time, such as from about ten seconds to about an hour, preferably about 10 minutes to about 30 minutes. The addition is conveniently carried out at ambient temperature, but the reagent may also be chilled or heated, if desired. After adding the crystallization solvent, the mixture may be mixed for an additional period of time to allow more ketal product to precipitate. Optionally, additional increments of crystallization solvent may be added and mixed for additional periods of time.

After the last addition of crystallization solvent, the mixture may be cooled and aged to upgrade yield and/or purity. For instance, aging may occur at a temperature of from about −25° C. to about 0° C., desirably −20° C. to about −10° C. for a period of about 5 minutes to about 8 hours, preferably about 10 minutes to about 2 hours. It was found that aging at about −20° C. to −18° C. provided excellent yields. The precipitated product may then be recovered via filtration. The wet cake collected on the filter may be washed one or more times with cold crystallization solvent. The collected product may then be dried.

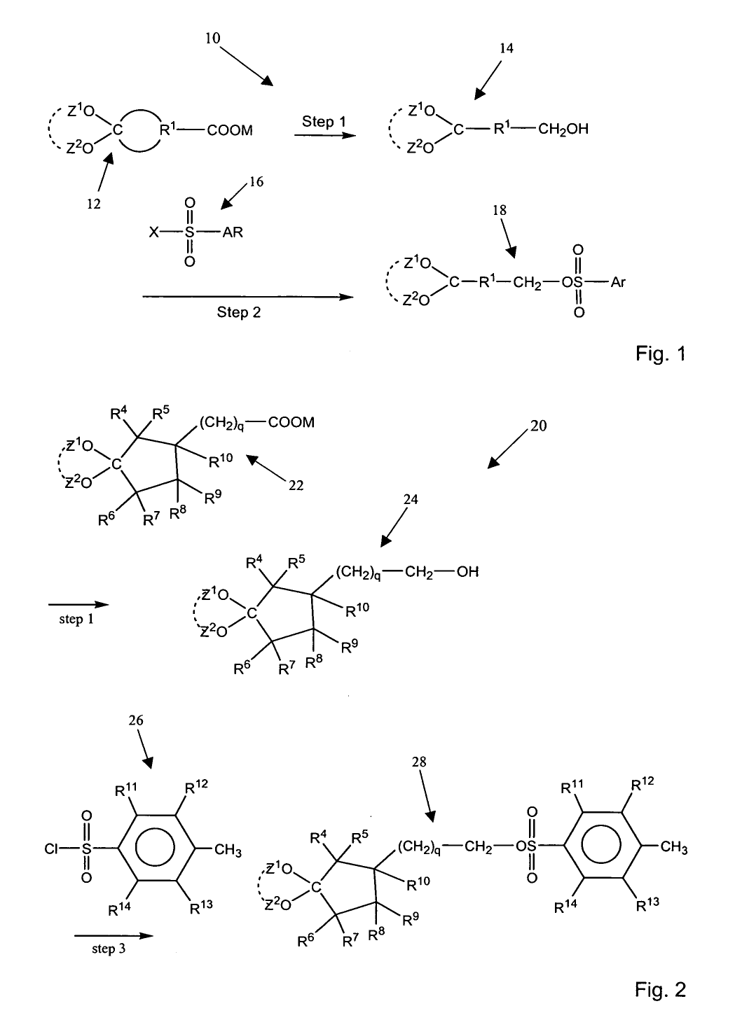

FIGS. 1 and 2 illustrate schematically how the two step synthesis scheme described herein may be used to obtain aromatic sulfonated ketals from a ketal acid (or salt or acid) starting material. FIG. 1 provides an illustrative reaction scheme 10 in which ketal acid 12 (or salt or ester) is converted to a ketal alcohol 14 via a reduction reaction in a first reaction step. This alcohol is then reacted with co-reactant 16 to form the aromatic sulfonated ketal 18 in a second reaction step. In this reaction scheme, the Z¹ and Z² are as defined above wherein the dotted line interconnecting these moieties indicates that these Z¹ and Z² moieties may be co-members of a ring structure. R¹ designates a trivalent linking moiety, and often, at least a portion of R¹ and the carbon atom attached to the OZ¹ and OZ² moieties form a cyclic structure that may be aliphatic and/or aromatic; and M is hydrogen, a monovalent organic substituent, or a suitable cation such as sodium, potassium, lithium, ammonium, an aromatic cation, combinations of these, and the like.

When M is a monovalent organic substituent, the monovalent organic substituent may comprise an aliphatic and/or aromatic hydrocarbyl moiety that may be linear, cyclic, or branched. In some embodiments, the embodiment of M in the form of a hydrocarbyl moiety includes from 1 to 10 carbon atoms, and often may be methyl or ethyl. In some modes of practice, M is a nitrogen-containing cation such as a compound of the formula N—(R^{o})₄⁺, wherein each R^{o} independently is hydrogen and/or an achiral or chiral monovalent moiety that may be substituted or unsubstituted, or linear, branched, or cyclic. In some embodiments, two or more of the R^{o} moieties may be co-members of a ring structure.

Preferably, at least one R⁰ moiety includes an aromatic ring linked to the N by a divalent linking group such as an alkylene moiety of 1 to 15 carbon atoms. For example, a particularly preferred nitrogen-containing moiety suitable for M is an aromatic cation that has the formula

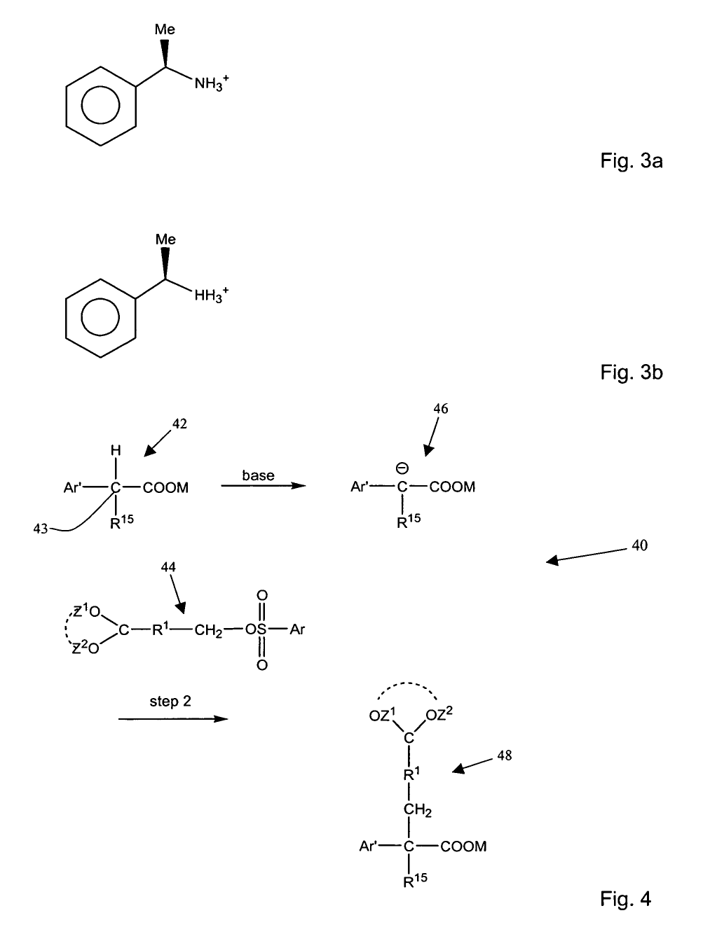

(Ar¹)ₚ—R²—N—(R⁰)_{q}⁺

wherein Ar¹ is a monovalent moiety comprising an aromatic ring; R² is a straight, branched, or cyclic alkylene moiety of 1 to 15, preferably 1 to 6 carbon atoms; p is 1 to 4, preferably 1; R⁰ is as defined above; q is 0 to 3; and p+q is 4. Preferably, the aromatic cation has the formula according to FIG. 1, wherein R^{o} and R² are as defined above; each Xⁿ is a monovalent substituent or co-member of a ring structure with another substituent; and r is 0 to 5, preferably 0. More preferably, the aromatic cation is an (R) and/or (S) chiral material having the formula according to FIG. 3a or FIG. 3b. In the context of a synthesis of the compound of Formula I, the aromatic cation is the (S) form according to FIG. 3a.

With respect to the co-reactant 16 of step 2, Ar is an aromatic moiety; r is 0 or 1; and X is a suitable leaving group such as a halide, especially chloride. The aromatic moiety may be any substituted or non-substituted (except for the oxo-hetero moiety when present as a substituent rather than a backbone constituent) moiety that includes at least one aromatic ring structure. The aromatic ring structure may be fused or non-fused with respect to other aromatic or aliphatic ring structures (e.g., as when two substituents of any such aromatic ring are co-members of a ring structure). The aromatic moiety optionally may incorporate one or more hetero atoms such as O, P, S, Si, N and/or the like as constituents and/or substituents of aromatic or aliphatic moieties incorporated into the aromatic moiety.

FIG. 2 shows a more preferred reaction scheme 20 in which a ketal acid (or salt or ester) 22 is converted to an alcohol 24 in step 1 via a reduction reaction and the alcohol 24 is then reacted with co-reactant 26 to form aromatic sulfonated ketal 28 in step 2. Z¹, Z², X and M are as defined above. Each of R⁴ through R¹⁴ independently is a monovalent substituent or a co-member of a ring structure such as H, alkyl, halide, aryl, aralkyl, combinations of these, and the like. Any of these may be linear, branched and/or cyclic and optionally may incorporate one or more hetero atoms such as O, P, S, Si, N, combinations of these, and the like. In one embodiment, the aromatic ketal sulfonate 28 is in the form of a tosylate, a brosylate, and/or a nosylate. Tosylates are preferred. Like tosylates, bromo-substituted compounds such as 4-bromobenzene sulfonate, also tend to be crystalline solids.

The principles of the present invention are beneficially used any time it is desired to prepare an aromatic sulfonated ketal from a ketal alcohol, which itself optionally may be derived from a ketal acid (or salt or ester). The aromatic sulfonated ketals offer many advantages. First, embodiments of the aromatic sulfonated ketals, particularly those incorporating tosylate moieties, are stable, solid, crystalline materials. This makes them much easier to purify and isolate than oils, especially for large scale production. Representative embodiments are much less toxic than iodide functional electrophiles, easing safety and handling concerns. The aromatic sulfonated ketals and their ketal alcohol and ketal acid precursors tend to be soluble in toluene, allowing the multi-step synthesis of the aromatic sulfonated ketals starting from the ketal acid (or salt or ester) and/or the ketal alcohol to occur in toluene, reducing the number of synthesis solvents that otherwise are involved with respect to synthesizing iodide functional counterparts. Toluene is widely available and inexpensive, which is a tremendous logistical and economic advantage for large scale production.

Also, the materials are strong electrophiles. This makes them useful intermediates in the synthesis of complex organic molecules such as pharmacologically important molecules. As one example, the aromatic sulfonated ketals are excellent electrophiles for alkylation reactions. One alkylation methodology of the present invention will be described in the illustrative context of reacting an aromatic sulfonated ketal electrophile with an aromatic acid or salt thereof to form stereoisomer products which have various utilities, such as intermediates in the synthesis of pharmacologically active molecules. The aromatic acid to be alkylated desirably includes a carboxylic acid moiety or salt thereof; a chiral carbon atom that has an H substituent and that is in an alpha position relative to the carboxylic acid (or salt or ester) moiety; and an aromatic moiety that is linked by a single bond or a linking group to the chiral carbon atom that is in an alpha position relative to the carboxylic acid (or salt or ester) moiety. The aromatic moiety and the carboxylic acid moiety, i.e., a —COOM moiety, may be as defined above. In the presence of a base, it is believed that the base tends to deprotonate the alpha carbon of the aromatic acid, while the aromatic moiety helps to stabilize the resultant anion. The aromatic sulfonated moiety of the ketal functions as a leaving group so that the ketal than attaches to the alpha carbon to accomplish the alkylation.

An illustrative alkylation reaction 40 is shown in FIG. 4. In step 1, aromatic acid 42 has an aromatic moiety Ar′ and an H linked to the alpha carbon 43. The R¹⁵ moiety may be any monovalent moiety or a co-member of a ring structure with a portion of the Ar moiety. In representative embodiments, R¹⁵ may be H, alkyl, halide, aryl, aralkyl, combinations of these, and the like. Any of these may be linear, branched and/or cyclic, chiral or achiral, and optionally may incorporate one or more hetero atoms such as O, P, S, Si, N, combinations of these, and the like. The aromatic acid 42 is reacted with the aromatic sulfonated ketal 44 in the presence of a base. The Z¹, Z², R¹, R and Ar moieties of the ketal 44 are as defined above. It is believed that the anion 46 forms as an intermediate. In step 2, the ketal 44 alkylates the anion 46 at the alpha carbon with respect to the —COOM moiety to form the product 48.

When the aromatic sulfonated ketal 44 is derived from the reaction of a ketal alcohol (e.g., schematically represented by the formula R′OH) and an aromatic sulfonyl halide such as tosyl chloride (schematically represented by the formula MePh-SO₂—Cl,), the resultant ketal tosylate may be represented by the formula R′O—SO₂-PhMe. The O linking the R′ to the S comes from the ketal alcohol. When this compound is reacted with a nucleophile such as anion 46, all three oxygens leave in the form of a tosylate group (toluene sulfonic acid anion) of the structure MePh-SO₂—O⁻. In this way, a carbon-carbon bond is formed between the alpha-carbon of the enolate anion intermediate and the carbon of the CH₂ group of the ketal-alcohol. This is how the oxygen is removed from the ketal-alcohol. The byproduct of the tosylation reaction is HCl which is scavenged by a suitable base (e.g., DABCO).

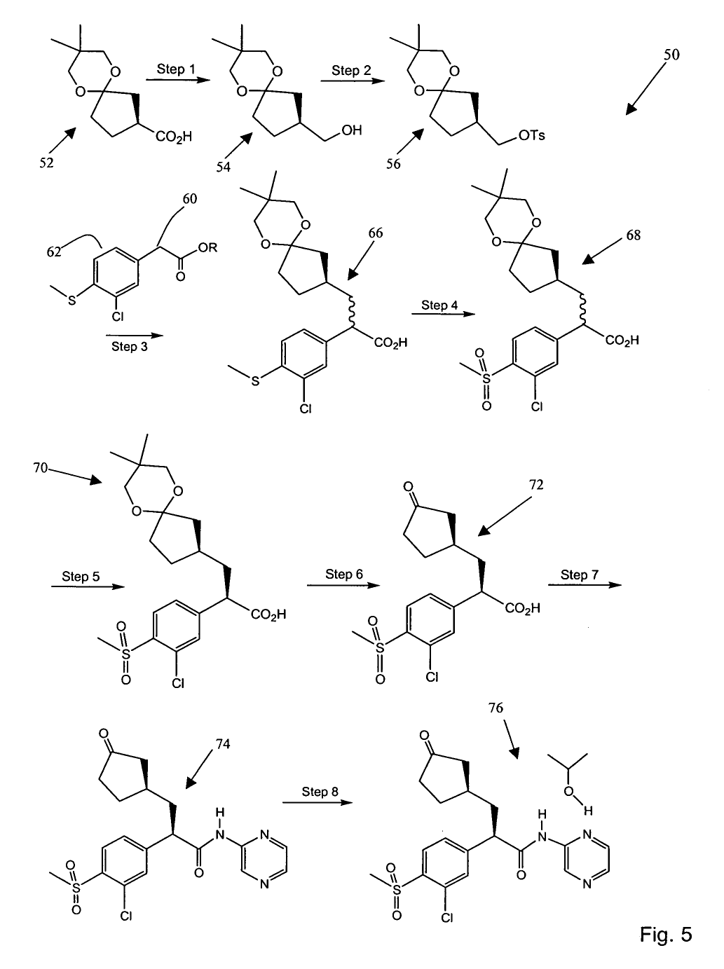

As another exemplary utility, the principles of the present invention may be used in the course of synthesizing pharmacologically active materials such as the compound of Formula 1. FIG. 5 shows one such illustrative scheme 50. In step 1, a chiral (S) ketal acid 52 is reduced to the corresponding chiral (S) ketal alcohol 54. The principles of the present invention are used to carry out this reduction reaction. This reaction is also described in co-pending U.S. Provisional Application No. 60/877,878, titled REDUCTION METHODOLOGIES FOR CONVERTING KETAL ACIDS, SALTS, AND ESTERS TO KETAL ALCOHOLS, bearing Attorney Docket No. RCC0031/P1, filed Dec. 29, 2006, in the name of Robert J. Topping, the entirety of which is incorporated herein by reference for all purposes The ketal acid 52 may be obtained from an S-MBA ketal salt precursor (not shown) using the techniques described in co-pending Application A. In step 2, the ketal alcohol 54 is converted to a tosylate 56 using techniques as described herein. Working examples below also describe this reaction. The —OTs (wherein Ts refers to tosylate) moiety has the formula

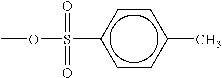

In step 3, the tosylate 56, a strong electrophile, is used to alkylate the alpha carbon 60 of the substituted, aromatic ester 62. This, too, may be accomplished using techniques as described herein and as included in the working examples below. The R group of ester 62 is desirably ethyl. The reaction is conveniently referred to as an alkylation inasmuch as the portion of the tosylate 56 that becomes directly linked to the ester 62 is the —CH₂— portion of the tosylate 56. An aromatic substituent 64 that is pendant from the alpha carbon 60 of the ester 62 is believed to help stabilize an anion intermediate that results when a base in the alkylation reaction medium helps to de-protonate the alpha carbon 60.

The reaction product of step 3 is a mixture of 2R,3′R and 2S,3′R epimers 66. The thio moiety of these is oxidized in step 4 to form the corresponding sulfonylated epimers 68. Step 5 involves subjecting the epimers 68 to an epimerization reaction to convert the 2S,3′R epimer to the desired 2R,3′R epimer 70. As an alternative option, this epimerization reaction may be carried out using the techniques as described in co-pending Application C inasmuch as the epimerization techniques of Application C may yield the desired epimer 70 in higher yield and purity.

Regardless of the epimerization technique used to carry out step 5, step 6 involves converting the ketal protecting group of epimer 70 to a ketone moiety to thereby form the sulfonylated, aromatic, ketone acid 72. In step 7, this acid 72 is reacted with a suitable co-reactant (not shown) to form the Formula I compound 74. In optional step 8, the Formula I compound 74 is converted to its IPA solvate form 76.

All patents, published patent applications, other publications, and pending patent applications (including both provisional and nonprovisional applications) cited in this specification are incorporated by reference herein in their respective entireties for all purposes.

The present invention will now be described with reference to the following illustrative examples.

### EXAMPLE 1

### Synthesis of Tosylate

### Salt Cleavage and (S)-Ketal-Acid Concentration

A 12,000 L glass-lined vessel was charged with 252.4 kg (752.4 mol) of (S)-Ketal-acid, (S)-MBA salt precursor of ketal acid 52 of FIG. 5 (this salt is described in Application A), followed by 1260 L (liters) toluene. The mixture (slurry) was cooled to 5° C. under nitrogen with agitation. To a 16,000 L glass-lined vessel was charged 212 L potable water followed by 318.0 kg 50% aqueous citric acid. The aqueous citric acid solution was cooled to 0° C. with agitation and then added to the ketal-acid salt slurry over 20 min while keeping the temperature of the reaction mixture below 5° C. The two-phase reaction mixture was warmed to 13° C. and allowed to settle for 30 min. The lower aqueous layer was separated. To the aqueous citric acid layer was added 504 L toluene. The two-phase mixture was stirred for 15 min at 14° C. and allowed to settle for 49 min at 14° C. The lower aqueous layer was separated. The two toluene extracts containing the intermediate (S)-ketal acid were combined and 84 L potable water was added. The two phase mixture was stirred for 17 min at 16° C. and the mixture allowed to settle for 60 min at 16° C. The lower aqueous layer was separated into a separate vessel.

To this aqueous solution was added 504 L toluene. The two-phase mixture was stirred for 20 min at 18° C. and allowed to settle for 30 min at 18° C. The lower aqueous layer was separated and combined with the aqueous citric acid solution and discarded. All of the toluene phases containing the (S)-ketal acid were combined and approximately 1,018 L of the toluene solution of the (S)-ketal-acid was transferred from the 12,000 L glass-lined vessel to a 2000 L Hastelloy vessel. Transfer of 1018 L of solution to the 2,000 L Hastelloy vessel provided for a significant amount of head space for the subsequent distillations to minimize the chance of bumping the batch into the vessel overheads.

The solution was concentrated via a feed-distillation under reduced pressure (30-40 mm pressure, vessel temperature ˜35° C. with a maximum bath temperature of 50° C.) until the volume of the (S)-ketal-acid solution reached 588 L. After the 12,000 L feed vessel is empty, the distillation was halted and the feed vessel rinsed with 84 L toluene to the distillation vessel. The distillation was then restarted and continued until the target volume was reached. The solution was sampled for Karl Fischer analysis and showed 0.007% contained water. The solution of the ketal-acid was then cooled to 10° C. prior to the feed to a VITRIDE solution (Rohm & Haas). VITRIDE is a commercial designation for an aluminumhydride reducing agent. The full chemical name of the VITRIDE material is Sodium Dihydro-bis-(2-Methoxyethoxy)Aluminate or SDMA. It is highly soluble in aromatic hydrocarbon solvents and is sold as a 70% solution in toluene.

### (S)-Ketal-Acid Reduction

A 2,000 L glass-lined vessel was charged with 487.8 kg 70% Vitride solution in toluene followed by 441 L toluene with agitation. Approximately 9 L of toluene is used to flush out the charging dip leg after the Vitride charge. After the toluene charge, a recirculation loop containing a ReactIR™ monitoring instrument was started to monitor the reduction. The diluted Vitride solution was cooled to <5° C. The pre-cooled ketal-acid solution was transferred to the Vitride solution through a 20-micron polishing filter and ¼″ mass-flow meter at a rate of 2.0 kg/min. A mass flow meter was utilized as a safety precaution to minimize the risk of adding the ketal-acid at a rate that would generate hydrogen faster than could be safely handled in the reduction vessel. The reaction is very exothermic but the heat and hydrogen flow is completely controlled by the ketal-acid feed rate. A maximum addition rate was 2.2 kg/min. A polishing filter was used to prevent any residual salts from plugging the relatively small mass flow meter. A total of 581 kg of ketal-acid solution was transferred (density 0.959 kg/L) The reaction temperature was maintained at <25° C. but with a target range of 20±5° C. throughout the ketal-acid addition. Running the reduction at a lower temperature (e.g. <10° C.) results in lower yields, presumably due to incomplete reduction. Maintaining ambient temperature for the reaction results in higher yields.

The vessel containing the ketal-acid solution was rinsed with 42 L toluene and the rinse transferred through the filter and mass-flow meter. The reduction reaction mixture was agitated for 70 min at 20-22° C. and sampled for reaction completion. The reaction was monitored by the ReactIR™ to check for the presence of the excess Vitride at the end of the reaction, but an HPLC sample was also taken to check for the presence of unreacted ketal-acid. To a 12,000 L glass-lined vessel was charged 596.8 kg 20% aqueous NaOH solution which was cooled to 2° C. with agitation. This quantity of 20% NaOH used for this batch (500 L, 600 kg) was determined by the minimum stirrable volume of the 12,000 L vessel used for the quench. The amount of NaOH can be reduced where practical concerns like this do not control. The recirculation loop used for the ReactIR™ was blown back into the reactor just prior to the quench.

The reaction mixture was then transferred to the aqueous NaOH solution through a ½″ mass flow meter while keeping the temperature of the quench mixture below 25° C. A maximum feed rate was set at 9 kg/min to control the hydrogen evolution. The addition time for this batch was 3 h with a maximum temperature of 16° C. (1,461 kg of reaction solution transferred).

The reduction reaction vessel was rinsed with 84 L toluene and the rinse transferred through the mass flow meter. The quench mixture was warmed to 16° C. and stirred for 1 h at 16-17° C. The agitation was stopped and the two-phase mixture allowed to settle for 1 h at 17° C. The lower aqueous layer containing the caustic aluminum salts was separated into another glass-lined vessel. To this aqueous solution was added 504 L toluene and the two-phase mixture stirred for 30 min at 21° C. and allowed to settle for 1 h at 21-22° C. The layers were separated and the two toluene layers containing the crude ketal-alcohol were combined followed by a 84 L toluene vessel rinse. To the aqueous layer was added 504 L toluene and the two-phase mixture stirred for 30 min at 18° C. and allowed to settle for 1 h at 18° C. The lower aqueous phase was separated and discarded (638 L for this batch).

The two toluene layers containing the crude ketal-alcohol were again combined followed by a 84 L toluene vessel rinse. To the total solution containing the intermediate ketal-alcohol was added 209 L potable water. The two-phase mixture was stirred for 38 min at 18-21° C. and allowed to settle for 1 h at 21° C. The water rinse serves to remove any residual salts, but also removes some of the 2-methoxyethanol liberated during the quench as well as the intermediate ketal-alcohol thus requiring toluene back-extractions to minimize yield loss. The lower aqueous phase was separated and to it was added 211 L toluene. The two-phase mixture was stirred for 30 min at 24° C. and allowed to settle for 1 h at 24° C. The toluene layer was recombined with the bulk ketal-alcohol solution followed by a 84 L toluene vessel rinse. To the aqueous layer was added 210 L toluene. The two-phase mixture was stirred for 40 min at 23° C. and allowed to settle for 1.7 h at 23° C. The layers were separated and the aqueous layer discarded (399 L for this batch). The toluene layer was recombined with the bulk ketal-alcohol solution followed by a 84 L toluene vessel rinse. At this point, the ketal-alcohol solution was sampled for 2-methoxyethanol which was then monitored during the subsequent feed distillation. Approximately 1,018 L of the toluene solution of the ketal-alcohol was transferred from the 12,000 L glass-lined vessel to a 2000 L Hastelloy vessel. The solution was concentrated via a feed-distillation under reduced pressure (20 mm minimum pressure, vessel temperature ˜30-35° C. with a maximum bath temperature of 50° C.) until the volume of the ketal-alcohol reached 320 L. The ketal-alcohol solution was held for 1 h and any second-phase water present removed prior to starting the feed distillation.

After the initial feed distillation was complete, the ketal-alcohol solution was sampled for 2-methoxyethanol and water content. It was necessary to add additional toluene and continue the distillation to remove the 2-methoxyethanol to an acceptable level. A total of three additional toluene charges were required (100 L, 150 L and 500 L) with the final distillation volume being reduced to 220 L. The final 2-methoxyethanol content was 0.022% relative to the ketal-alcohol. Since the ketal-alcohol solution was to be eventually transferred back to the 12,000 L vessel for the tosylation reaction, no vessel rinse was performed during the distillation.

### Tosylation

The toluene solution of the intermediate ketal-alcohol was transferred to a 12,000 L glass-lined reactor followed by a 150 L toluene rinse. The 2,000 L Hastelloy reactor was vacuum dried and to it was charged 105.9 kg (944.0 mol) 1,4-diazabicyclo[2.2.2]octane (DABCO) followed by 605 L toluene. The mixture was stirred for 1.2 h at 15-16° C. until the solids were dissolved. The DABCO solution was combined with the solution of the ketal-alcohol followed by a 42 L toluene vessel rinse. The vessel used for the DABCO solution make-up was again vacuum dried and to it charged 162.1 kg (850.2 mol) p-toluene sulfonyl chloride (tosyl chloride) followed by 542 L toluene. The mixture was stirred for 15 min at 10-16° C. to dissolve the solids (dissolution is endothermic) and then cooled to 2° C. The solution of tosyl chloride was then transferred to the solution of the ketal-alcohol and DABCO while keeping the reaction temperature <10° C. (addition performed over ˜3 h with a temperature range of −2 to +6° C.). To the vessel containing the tosyl chloride was added 43 L toluene as a vessel rinse. The reaction was stirred for 1 h at 3 to 4° C. and sampled for reaction completion (HPLC). The reaction completion showed 1 mg/mL ketal-alcohol remaining with excess tosyl chloride still present.

While the reaction completion sample was been analyzed, a 16,000 L glass-lined vessel was charged with 700 L potable water followed by 63.8 kg (759 mol) sodium bicarbonate. The mixture was stirred at ambient temperature to dissolve the solids. Once the tosylation reaction was deemed complete, the reaction mixture was added to the aqueous bicarbonate solution over 2 h at ambient temperature (jacket temperature setpoint of 20° C.) followed by 85 L toluene as a vessel rinse. The two-phase mixture was stirred for 2.3 h at 25-28° C. to hydrolyze the excess tosyl chloride and sampled for reaction completion (tosyl chloride not detected). The mixture was allowed to settle for 1 h at 29° C. and the lower aqueous layer separated. To the upper toluene layer was added 504 L potable water. The two-phase mixture was stirred for 30 min at 27° C. and allowed to settle for 1 h at 27° C. The lower aqueous layer was separated, combined with the sodium bicarbonate extract and discarded. The toluene solution of the Step 6 product was filtered through a 10-inch, 20-micron filter to a 12,000 L glass-lined reactor followed by 127 L toluene used as a vessel rinse. The filtered toluene solution was allowed to settle for at least 30 min followed by removal of any second phase water that was present before starting the subsequent feed distillation.

### Crystallization & Isolation

Approximately 1,018 L of the toluene solution of the (S)-chiral tosylate was ransferred to a 2,000 L Hastelloy vessel. The solution was concentrated via a feed-distillation under reduced pressure (20 mm minimum pressure, vessel temperature ˜30-35° C. with a maximum bath temperature of 50° C.) until the volume of the (S)-chiral tosylate solution reached. 353 L (no toluene rinse of the 12,000 L vessel was performed). The final strip volume prior to the heptane addition is important inasmuch as too much toluene will result in a lower product yield due to losses to the mother liquors.

Approximately half of the product solution was transferred to a 2,000 L glass-lined vessel (180 kg, density 1.07 kg/L) with the other half transferred back to the 12,000 L glass-lined feed vessel for storage. The 2,000 L glass-lined vessel (used as the Heinkel feed vessel) was too small to accommodate crystallizing the entire batch. It was therefore split and the crystallization performed in two parts. The batch was transferred through a mass flow meter to accurately determine how much of the batch was contained in each part. In this case, approximately 220 kg was contained in the second part necessitating that additional heptane above the standard charge be added for the second part crystallization.

To the Hastelloy distillation vessel was added two separate portions of 15 L toluene as a line rinse to each of the two glass-lined vessels. To the 2,000 L glass-lined vessel containing the first half of the batch was added 713.5 kg n-heptane at ambient temperature (20±5° C.) to crystallize the product. To each drum of n-heptane was added 8-10 drops of Octastat 5000 to increase the solvent conductivity. The charge rate of the n-heptane was limited to <8 kg/min. The product crystallization occurs during the heptane addition.

After the heptane addition and the product crystallization had occurred, the product slurry was cooled to −15° C. over 5.5 h and allowed to stir for 1 h at −15° C. The cool-down rate after the crystallization is not critical since the batch crystallizes during the heptane addition at ambient temperature. The product was isolated using a Heinkel and washed with pre-cooled n-heptane (<−10° C.) to give 108.1 kg product wet cake. As necessary, the mother liquors were used to rinse product from the crystallization vessel after the initial filtration sequence, especially for the second half isolation. After isolation was completed, the wet cake was loaded to a double-cone dryer and drying initiated (35° C. maximum bath temperature, full vacuum) while the second half of the batch was crystallized.

The second half of the batch was transferred from the 12,000 L glass-lined vessel to the 2,000 L glass-lined vessel used for the first half crystallization followed by 81.1 kg n-heptane as a vessel and line rinse. To the product solution was added an additional 790.6 kg n-heptane at 20±5° C. to crystallize the second half of the batch. After the heptane addition and the product crystallization had occurred, the product slurry was cooled to −15° C. over 10 h and allowed to stir for 1 h at −15° C. to −18° C. The product was isolated using a Heinkel and washed with pre-cooled n-heptane (<−10° C.) to give 140.2 kg product wet cake (248.3 kg total). After isolation was completed, the wet cake was loaded to the double-cone dryer containing the first half of the batch and drying restarted (35° C. maximum bath temperature, full vacuum). The product was dried until an LOD of <1.0% was achieved (0.00% LOD obtained on batch, LIMS 38-478512). The product was discharged from the dryer into double poly-lined fiber packs to give 224.1 kg (632.2 mol, 84.0% yield) of the (S)-chiral tosylate. A stirred sample of the mother liquors (˜3,100 L) was obtained which showed that ˜17.5 kg (49.5 mol, 6.6% yield based on 5.7 g/L assay of mother liquor sample) product was contained in the mother liquors.

### EXAMPLE 2

### Reaction corresponding to Steps 3 and 4 of FIG. 5

A mixture of 268 g THF and 177.7 g (1.00 equiv) of ethyl(3-chloro-4-(methylthio)phenylacetate were slowly added to a cold (<−15° C.) 20% solution of potassium tert-butoxide in THF (415.5 g, 1.02 equiv) and allowed to react over 2 hours at −15° C. to form a potassium enolate. A 1:1 solution mixture of 256.1 g (1.00 equiv) of the (S)-(8,8-dimethyl-6,10-dioxaspiro[4.5]decan-2-yl)methyl 4methylbenzenesulfonate and 321 g THF was transferred slowly to the cold enolate reaction mixture solution. The alkylation reaction mixture was stirred at <0° C., warmed to 40° C. and then held at 40° C. until the reaction was complete. The THF was distilled off under vacuum and the resulting ester product extracted into 629 g of MTBE and 445 mL water. The bottom aqueous layer was extracted with another 100 g MTBE. The resulting two MTBE/product layers were combined.

The resulting intermediate alkylation product ester as a MTBE solution was directly utilized for a hydrolysis reaction by adding 69 g (1.2 equiv) of 50% aqueous sodium hydroxide. The mixture was heated to 50° C. and stirred until the reaction was complete. The. MTBE was removed by distillation under vacuum. The product mixture was extracted into water by adding 613 g water and 612 g toluene. The bottom aqueous product layer was extracted again by adding 612 g toluene to remove any remaining by-products. The bottom aqueous product layer was pH adjusted using 35 g of a 50% citric acid solution to a pH of 8.5-9.0. The aqueous product layer was concentrated under vacuum to remove all residual toluene. This water mixture was carried into the subsequent oxidation reaction.

A solution of 4.8 g (0.02 equiv) sodium tungstate dihydrate catalyst in 12 g water was added to the reaction mixture. The reaction mixture was cooled to <15° C. and 209 g (1.2 equiv) of 30% aqueous hydrogen peroxide was added, maintaining the reaction mixture below 45° C. The mixture was pH adjusted to approximately 7.5 by adding small amounts (˜1-5 g) of a saturated aqueous sodium bicarbonate solution, if necessary. The reaction was stirred at 20° C. until the reaction was complete. The peroxide mixture was quenched using an aqueous solution of sodium sulfite. The quenched reaction mixture was then extracted by adding 407 g isopropyl acetate. The top organic layer was separated to remove by-products. The bottom aqueous product layer was pH adjusted to 4.7 using 140 g of a 50% solution citric acid and extracted by adding 610 g isopropyl acetate. The bottom layer was separated and extracted again with 558 g isopropyl acetate. The combined organic/product layers were distilled under vacuum to remove residual water. The resulting product slurry was heated to 65° C. with an additional 370 g of isopropyl acetate. The mixture was crystallized by slowly adding 498 g n-heptane. The slurry was concentrated under vacuum and an additional 632 g n-heptane added. The mixture was concentrated and slowly cooled to 10° C., aged, filtered, washed with n-heptane and dried to produce 229.7 g (73.8% yield) of (R)-2-(3-chloro-4-(methylsulfonyl)phenyl)-3-(8,8-dimethyl-6,10-dioxaspiro[4.5]-decan-2-yl)propanoic acid as a dry solid powder.

### EXAMPLE 3

### Epimerization Reaction, Dissolution, Recrystallization, Filtering and Drying

### Sodium Salt Formation

A 2000 L glass-lined reactor (vessel 1) was charged with 99.8 kg (232 mol, 1.00 equiv) of the reaction product 68 shown in FIG. 5 followed by 165.5 kg of denatured, 2B-3 ethanol. The mixture was stirred at 20° C. for 10 min. A solution of 112.5 kg 21% sodium ethoxide in ethanol was charged to vessel 1 followed by a line rinse of 5.1 kg denatured ethanol, 2B-3.

### Chiral Epimerization

The mixture was heated to 65° C. and stirred for ˜6 hours. The mixture was then cooled to 55° C. and sampled by chiral HPLC to determine the diastereomer ratio. After the age with sodium ethoxide in ethanol the percentage of the undesired isomer was expected to be <20% (2S,3′R) relative to the (2R,3′R) isomer by chiral HPLC analysis and, indeed, in the lab, 14.7% to 18.5% (2S,3′R) was observed. This is as far as the epimerization can be taken in pure ethanol without significant production of aryl ethoxy and des-chloro impurities. While waiting for sample results, the mixture was heated back to 65° C.

While maintaining the vessel contents at 65° C., 573.1 kg of heptane was charged and the mixture stirred and aged for 2 h at 65° C. To add the heptane, ethanol is exchanged via a vacuum feed-strip with heptane and the reaction mixture aged for a minimum of a 2 hours. The mixture was then cooled to 55° C. and sample. After the addition and age with n-heptane, the ratio of the (2R,3′R) to (2S,3′R) isomers was monitored along with the production of aryl ethoxy and des-chloro impurities using the chiral HPLC method. The bath temperature was set at 75° C. (the mixture refluxes at approximately 67° C.) and the reaction mixture was concentrated by atmospheric distillation to ˜450 L. The mixture was then cooled to 55° C. and sampled for final epimerization completion. After the distillation of n-heptane/ethanol, the percentage of the undesired isomer was expected to be <8% (2S,3′R) relative to the (2R,3′R) isomer by chiral HPLC analysis, and indeed 5.5 to 7.5% was observed. This is as far as the epimerization can be taken without significant production of aryl ethoxy and des-chloro impurities.

The mixture was cooled to 20° C. and 7.0 kg (117 mol, 0.5 equiv) of acetic acid was charged to another vessel, hereinafter vessel 2, followed by a line rinse of 2.0 L methanol. To vessel 2 was then charged 250.0 L methanol and the mixture stirred for 15 min. The mixture in vessel 2 was then transferred to vessel 1 while maintaining the vessel 1 contents at 20 (±5)° C. The reaction mixture was stirred for 15 min at 18° C. The mixture was then sampled to ensure the pH was between 6 and 8. The actual pH was 7.5. To vessel 2 was next charged 750 L methanol which was heated to 40° C. The reaction mixture in vessel 1 was atmospherically distilled while continuously charging methanol from vessel 2 through a ¼″ mass flow meter to maintain a constant volume in vessel 1. The mixture refluxes at about 57° C. About 880 L of distillate is collected. To vessel 2 was then charged 479.8 kg isopropyl alcohol which was heated to 50° C. The reaction mixture in vessel 1 was atmospherically distilled while continuously charging isopropyl alcohol from vessel 2 through a ¼″ mass flow meter to maintain a constant volume in vessel 1. The mixture will begin to reflux at ˜57° C. and this will increase to 71° C. The resulting slurry was slowly cooled over 2 h to 20° C. followed by aging at 20° C. for 1 h. The slurry was then sampled for final recrystallization completion. The slurry sample was filtered and the mother liquors analyzed for ratio of (2R,3′R) to (2S,3′R) isomers by chiral HPLC for comparison to reference batches (26.3 to 32.0% area norm (2R,3′R) was observed) to ensure the correct volume had been reached to obtain a good yield of the product. The result was 32% area norm for the (2R,3′R) isomer and 68% area norm for the (2S,3′R) isomer by chiral HPLC analysis, which was consistent with reference batches.

### Isolation and Drying

To a Heinkel rinse vessel (vessel 3) was charged 272.9 kg isopropyl alcohol. The product slurry was filtered through the Heinkel centrifuge filter unit. Each spin was rinsed with a small quantity of the isopropyl alcohol from vessel 3. Each spin was initially filled with 3-5 kg of slurry and the product washed with ˜1 kg of isopropyl alcohol which resulted in about 1.5-2.0 kg of wet cake per spin. The combined isolated wet cakes were transferred to Krauss-Maffei conical dryer and dried under vacuum for 18 h at 40-45° C. to produce 94.7 kg (209 mol, 90.3% yield) of (2R, 3′R)-sulfone ketal acid, sodium salt [(2R,3′R)-9)] isolated as a dry powder. The isolated material showed excellent purity by chiral HPLC and area normalized purity of 98.91%. However, the assay purity was only 93.6 wt % due to the presence of residual sodium acetate produced during neutralization with acetic acid. This salt was effectively removed in a subsequent hydrolysis step.

Other embodiments of this invention will be apparent to those skilled in the art upon consideration of this specification or from practice of the invention disclosed herein. Various omissions, modifications, and changes to the principles and embodiments described herein may be made by one skilled in the art without departing from the true scope and spirit of the invention which is indicated by the following claims.

What is claimed is:

1. An aromatic ketal sulfonate, comprising:
a sulfonyl moiety of the formula —S(O)(O)—, wherein there is a double bond between each oxygen and the sulfur atom;
a ketal moiety linked to the sulfonyl moiety by a first linkage; and
an aromatic moiety coupled to the sulfonyl moiety by a second linkage.

2. A method of making an aromatic, sulfonylated ketal, comprising the steps of:
providing a ketal alcohol (or amine); and
reacting the alcohol (or amine) with a co-reactant comprising a source of an aromatic sulfonyl moiety under conditions effective to convert the alcohol moiety of the ketal to an aromatic sulfonyl moiety.

3. The method of claim 2, wherein said providing step comprises reducing a ketal acid (or salt or ester thereof) to form the ketal alcohol.

4. A method of using an aromatic ketal sulfonate, comprising the step of reacting the aromatic ketal sulfonate with an aromatic ester in the presence of a base, wherein the aromatic ester comprises:
a carbon atom that is in an alpha position relative to a —C(O)— moiety;
an aromatic moiety is linked to the alpha carbon atom; and
wherein at least one remaining substituent of the alpha carbon atom is H.

5. A method of using an aromatic ketal sulfonate, comprising the step of using the aromatic ketal sulfonate in a synthesis scheme that prepares the compound [chemical structure]
